(19) United States
(12) Patent Application Publication
(10) Pub. No.: US 2007/0190165 A1
(43) Pub. Date: Aug. 16, 2007

(54) Tissue-specific basement membrane gels

(76) Inventors: Eric M. Brey, Chicago, IL (US); Shiri Uriel, Chicago, IL (US); Edwardine Labay, Des Plaines, IL (US); Ralph R. Weichselbaum, Chicago, IL (US)

Correspondence Address: Pauley Petersen & Erickson, Suite 365, 2800 West Higgins Road, Hoffman Estates, IL, 60169, US

(21) Appl. No.: 11/585,353

(22) Filed: Oct. 23, 2006

Related U.S. Application Data
(60) Provisional application No. 60/728,910, filed on Oct. 21, 2005.

(51) Int. Cl. A61K 35/12

(52) U.S. Cl. 424/520

(57) ABSTRACT

Methods are provided for basement membrane extraction and gellation whereby a biologically active, stable, three dimensional gel can be induced within five minutes or less with basement membrane components from substantially any tissue selected by the preparer. The three dimensional gel can further be substantially free of cellular contaminants. Thus, a wide range of diagnostic and therapeutic uses for such gels can be made available.

This patent application claims priority from U.S. Provisional Application 60/728,910, filed 21 Oct. 2005. The co-pending provisional application is hereby incorporated by reference herein in its entirety and is made a part hereof, including but not limited to those portions which specifically appear hereinafter.

### BACKGROUND OF THE INVENTION

1. Field of the Invention

The present invention provides improvements to the manufacture of biologically active gels. Specifically, the present invention is related to a method of allowing a preparer to select substantially any tissue and quickly replicate or duplicate its basement membrane in a three dimensional gel.

2. Discussion of the Related Art

Research has shown that basement membranes play an important role in tissue biology and function. Basement membranes influence cell migration, proliferation, resistance to therapeutics, and differentiation of cells. In addition, basement membranes stimulate angiogenesis and can guide stem cells towards a particular cell phenotype depending on their source. In addition, other basement membrane molecules, and fragments derived from them, are regulators of biological activities in a tissue-source specific manner. However, all basement membranes are not alike.

The known art has largely focused on purified basement membrane components (collagens, laminins, fibronectin, etc.). Thus, basement membrane simulations made according to such art will lack a full complement of basement membrane molecules or structural details and may lack tissue specific components as required for various research.

Other attempted basement membrane simulations may be manufactured from synthetic components but will lack a natural substrate having a full complement of basement membrane components in a natural ratio and thus may be of limited therapeutic applicability.

One known basement membrane complex, as disclosed in U.S. Pat. No. 4,829,000 to Kleinman et al., and commercially available as MATRIGEL, from BD Biosciences of San Jose, Calif. USA 95131 teaches membrane components extracted from a spontaneous mouse tumor, the Engelbreth-Holm-Swarm (EHS) tumor, and has become an important tool for studying both two dimensional and three dimensional cell-matrix interactions. It is used routinely in labs throughout the world for studies of basement membrane-cell interactions, angiogenesis (in vitro and in vivo), tumor invasion, and as a scaffold for tissue engineering applications.

However, MATRIGEL does not accurately reflect the diversity of basement membrane proteins of most tissues. While MATRIGEL is frequently used as a general tool for basement membrane-cell interactions, it primarily comprises laminin-1 and collagen type 4. Laminin-1 is not present at high quantities in most adult tissues. In addition, many other laminins and basement membrane components that are not present in MATRIGEL, such as other laminin subtypes, may play a vital role in the pathology and physiology of other tissues. Also, decellularization is not taught by the U.S. Pat. No. 4,829,000 patent, which may lead to therapeutic complications.

Another method, as taught in U.S. Pat. No. 5,147,782 to Brocks et al., extracts basement membrane components from human and animal tissues in the presence of a chelating agent. However this approach does not result in three dimensional gels and also does not remove cellular contaminates which may have adverse affects.

Two synthetic matrices are commercially available: PURAMATRIX from BD Biosciences of San Jose, Calif. USA 95131, and CYTOMATRIX, from Cytomatrix, LLC of Woburn, Mass. USA 01801. While these are interesting products from certain standpoints, they consist of synthetic materials and other materials may have to be added to the products to make them biologically active.

### SUMMARY OF THE INVENTION

What is needed in the art is a fundamental difference of approach whereby the user, e.g., a clinician or scientist or engineer, is allowed to select the tissue for replicating or duplicating the basement membrane thereof. In order to adequately understand tissue specific basement membrane-cell interactions, basement membrane components, sometimes referred to herein as proteins, are needed that adequately reflect the diversity of many different tissues in the body. The present invention allows for the isolation of basement membrane components and rapid gellation into a stable three dimensional gel for replicating or duplicating the tissue specific basement membrane from substantially any tissue sample, e.g., from any species, any pathology, etc. Thus, the present invention will allow researchers or clinicians to have a tool for studying a basement membrane from tissues or pathologies of their specific interest. In addition, gels made according to the present invention may have promise for use in regenerative medicine, e.g., for their ability to guide stem cells and other cells towards a tissue-specific phenotype.

The general object of this invention can be obtained, at least in part, through a method of obtaining a gel structure including a protein extract from a tissue. The method includes selecting a tissue for extraction of basement-membrane proteins; obtaining a solution of the basement membrane components from the tissue; and lowering the pH of the solution of the extracted basement membrane components or adding cells, such as fibroblasts, to the solution extracted basement membrane components to gel the extracted basement membrane. In one embodiment of this invention, the pH is lowered to below about 5, and more desirably is lowered to about 4.

The invention further comprehends a method of obtaining a gel structure that includes: selecting a tissue for extraction of basement-membrane proteins; removing cells of the selected tissue to achieve a decellularized basement membrane free of cellular contaminants; extracting basement membrane components from the decellularized basement membrane; and gelling the extracted basement membrane. Gelling the extracted basement membrane can be obtained by lowering the pH of a solution of the extracted basement membrane to below about 5, increasing the solution temperature above about 30° C., or by addition of cells to a solution of the extracted basement membrane. Increasing the temperature is particularly useful when the concentration of the basement membrane in the solution is greater than about 5 mg/ml.

The invention still further comprehends a method of obtaining a gel structure that includes: selecting a tissue for extraction of its basement-membrane proteins; homogenizing the basement membrane in a salt solution containing a protease inhibitor; extracting basement membrane components of the homogenized basement membrane in a high molar urea solution; and gelling the extracted basement membrane components in the urea to attain a gel structure by lowering the pH of the solution or adding cells to the solution.

The present invention provides both methods for extracting basement membrane components from natural tissues and methods for inducing the basement membrane components to assemble into stable three dimensional gels with nanostructure similar to the basement membrane as found in vivo. Endothelial cells, fibroblasts, and tumor cells have been shown to readily attach to the natural gels with three dimensional structure of the present invention.

In a first process of the invention, a selected tissue source is decellularized and its basement membrane components are extracted from the selected tissue source, e.g., normal or abnormal mammalian tissue samples. In a second process of the invention, the extracted basement membrane components are induced to form a stable three dimensional gel with a nanostructure similar to basement membrane found in vivo. A major advantage of the present invention is that the basement membrane components in the gels are specific to the source of the tissue. “Source,” as used herein may denote, e.g., the species, tissue or organ type, or pathology, of the tissue.

The extraction procedure in one embodiment involves the steps of decellularization of the selected tissue followed by extraction of the basement membrane components from the decellularized tissue. Decellularization can be performed by brief treatment of the tissue with a chemical (e.g., EDTA) or enzymatic treatment (e.g., dispase), or a gentle grinding, either or both of which would be followed by pressing the cells through a cell sieve or washing the cells off the tissue. If cellular contamination is not an issue, such as where the tissue is to be used autologously, the decellularization step may be omitted in certain embodiments. The selected tissue, whether decellularized or not, is then ground in a high salt concentration solution in the presence of protease inhibitors to minimize loss of basement membrane proteins into the solution. The basement membrane components are then extracted by a soaking, which may be accompanied with stirring, e.g., in high urea solution for an effective time (e.g., 10-24 hours). The extracted basement membrane components are then gelled into a three dimensional matrix by control of one or more of the concentration, pH, and temperature of the extracted-components solution. In addition, or in the alternative, a sufficient number of cells may be added to the extract solution of the basement membrane components and thereby induce gellation in the absence of any other environmental changes.

The present invention thus provides a replicated or duplicated basement membrane via a biologically active gel, with no cellular contaminants if desired, and thus having a tissue specific composition substantially of the same basement membrane components in the same ratio as the originally selected tissue's basement membrane. While ultimate verification may be unattainable due to the nature of biological investigation, it is believed that the three-dimensional gels of the present invention should have all, or substantially all, of the basement membrane proteins in substantially the same ratio as the original tissue basement membrane thus providing a substantially full complement of natural proteins for the replicated or duplicated basement membrane of the three dimensional gel.

The present invention further desirably allows that no extraneous proteins are used or needed in the gellation process. Because the gel of the present invention is essentially a duplicated, or closely replicated, version of the selected tissue basement membrane, in some instances the therapeutic use of the gel can be enabled without fear of rejection reactions. Desirably, the present invention provides the use of gels for scientific and therapeutic applications specific to the tissue or cell type being investigated or treated.

In an exemplary embodiment, the method of deriving a basement membrane complex extract from any one of a variety of naturally occurring tissue sources, and gelling the extract into a stable three-dimensional gel structure, can comprise selecting a tissue for extraction of its basement-membrane proteins; and removing substantially all cells of the tissue to achieve a decellularized basement membrane free of cellular contaminants if desired. Following cell removal, the decellularized basement membrane is homogenized in a high salt solution in the presence of protease inhibitors. The absence of cellular components in the homogenized decellularized basement membrane can then be confirmed, if desired. The natural basement membrane components are then extracted from the homogenized decellularized basement membrane by incubation in a high molar urea solution overnight.

The extracted substantially-full complement of natural basement membrane components are desirably concentrated to greater than about 0.1 mg/ml, and more desirably greater than about 1 mg/ml, in a urea solution by selection of the ratio of urea volume to weight of the original tissue sample. The extracted natural basement membrane components, unique to the tissue source, are then gelled without addition of extraneous and/or synthetic proteins or crosslinking agents to rapidly, e.g. within 5 minutes, attain a three-dimensional gel structure by one or more of the steps of either altering the pH if the concentration of basement membrane components is below a critical level, or raising the temperature if the concentration of basement membrane components is above the critical level. For a broad range of concentration levels of the extract solution, gels can be induced by the addition of a sufficient number of cells to the extract solution without a change in pH or temperature.

The gel can be obtained within about 5 minutes of adjusting the pH (e.g., lowering below about 5), between about 2 to 3 hours by adjusting the temperature (e.g., increasing to above about 30° C.), or between about 30 to 60 minutes by cell addition (e.g., adding fibroblasts). The pH of the three-dimensional gel is then maintained, stabilized, or increased to about 7.0 to about 7.4; whereby a three-dimensional gel stroma substantially free of cellular contamination, if desired, and substantially similar to the morphology and protein structure of the original tissue basement membrane is obtained.

Words of degree, such as “about”, “substantially”, and the like are used herein in the sense of “at, or nearly at, when given the processing, design, and material tolerances inherent in the stated circumstances” and are used to prevent the unscrupulous infringer from unfairly taking advantage of the invention disclosure where exact or absolute figures are stated as an aid to understanding the invention.

As used herein, references to “decellularized” or “decellularization” are to be understood to refer to a biostructure (e.g., a tissue), or a method of obtaining a biostructure, from which the majority of the cellular content has been removed leaving behind an intact mostly acellular infra-structure.

Organs and tissues are made up of cellular components and extracellular components. The supporting fibrous network of the organ is the stroma. Most organs have a stromal framework composed of insoluble proteins that make up a network of connective tissue which supports the tissue and its cells. The process of decellularization removes the cells, leaving behind the complex three-dimensional network of extracellular proteins. This connective tissue infrastructure includes all extracellular matrice, including secreted proteins, stromal matrices, and basement membranes. Decellularized biostructures can be rigid, or semi-rigid, having an ability to alter their shapes. The removal of cell content via a decellularization procedure according to this invention, prior to basement membrane extraction, reduces the chance of intracellular contents contaminating the extracted proteins. This will reduce chance of rejection when used in research animal models or in clinical application and will reduce the chance of non-basement membrane specific interactions when used in vitro. Examples of decellularized tissues useful in the present invention include, but are not limited to, the dermis, tumor samples, adipose tissues, aorta, islet cells, and pancreas.

### BRIEF DESCRIPTION OF THE DRAWINGS

These and other objects and features of this invention will be better understood from the following detailed description taken in conjunction with the drawings wherein:

FIG. 1 shows three graphs of gellation versus time for rat dermis, U87 (glioblastoma), and SCC-61 (squamous cell carcinoma), respectively, following a change in pH.

FIG. 2 shows a scanning electron microscope image of a gel from extracted U87 glioblastoma basement membrane proteins, where the white bar represents one micron.

FIG. 3 shows scanning electron images of a gel (A) and an image from literature of intact basement membrane (B).

FIG. 4 shows a transmission electron microscope image of an U87 glioblastoma gel showing diffusively flocculent structure similar to that seen in basement membrane structure in vivo (A) and an image from the literature showing the structure of a basement membrane in vivo (B

FIG. 5 shows PAS stains of original tissue (A) and extracted gels (B). Round nuclei are seen only in the original tissue, but the matrix structure is similar in both tissues. The dark spot in (B) is a sectioning artifact.

FIG. 6 shows 16% SDS PAGE gels of extracts from MATRIGEL (A) and an extracted gel (B) according to one embodiment of the present invention.

FIG. 7 shows Western blots for beta actin on dispase-treated and untreated extractions. Lane 5 is the molecular weight marker.

FIG. 8 shows a collagen type IV Western blot indicating the presence of collagen IV in the irradiated glioblastoma tumor (U87) extract and rat dermis basement membrane extraction.

FIG. 9 shows a Western blot analysis of gamma 1 chain of laminin in U87 and SCC-61 basement membranes extractions with MATRIGEL in lane 1 for a control. Lane 6 is the molecular weight marker.

FIG. 10 is a table summarizing ELISA results identifying basement membrane components in basement membrane gels from various tissues. MATRIGEL is shown as a control for differences between our gels and the industry standard.

FIG. 11 is a graph showing absorbance readings, useful in indicating gel formation, upon the addition of cells.

FIG. 12 is a graph showing the results of growing fibroblasts and human umbilical vein endothelial cells (HUVECS) on extracts of dermis basement membranes (BM). When used as a thin coating, the cells grew rapidly, while as a thick 3D gel, the cells differentiated and became quiescent (viable without growth).

### DESCRIPTION OF PREFERRED EMBODIMENTS

Discussion of the present invention will be given herein with respect to specific exemplary embodiments that are in some cases arbitrarily or specifically designated for explanatory purposes. It will be appreciated by the person having ordinary skill in the art that aspects of the present invention may be arranged in a variety of ways and that other nomenclature than is used herein might be applied without doing violence to the spirit of the present invention. The invention illustratively disclosed herein suitably may be practiced in the absence of any element, part, step, component, or ingredient which is not specifically disclosed herein.

The present invention generally provides a method of isolating and quickly replicating or duplicating a substantially complete basement membrane from substantially any tissue as selectable by the preparer. The isolated basement membrane is solubilized and reconstituted as a stable three-dimensional gel. The gel maybe used for structural, therapeutic and diagnostic applications. The present invention can easily be scaled for high throughput production and can be applied to various tissue sources.

Gellation to attain the three dimensional gels can be induced through control of the concentration of the basement membrane extract, temperature, pH, and/or the addition of cells to the basement membrane extract. As the extracts of the various body tissue basement membranes are extremely variable in the number and concentration of the components that allow the creation of three dimensional gels; the gellation steps of the present invention provide a method to utilize essentially any tissue type for creating the replicated or duplicated basement membranes. Contamination of these gels with cellular proteins is minimized, if desired, by removing the cells prior to extraction of the proteins in some embodiments. Gels prepared according to the present invention can provide useful tools for therapeutic screening since they are tissue specific and can properly replicate or duplicate the in-vivo basement membrane environment. The various end uses such as exemplified herein are enabled due to the diversity of selectable stable three dimensional gels available from the present invention and the fact that the stable three dimensional gel replicates or duplicates the in vivo basement membrane to a high degree.

While ultimate verification may be unattainable due to the nature of biological investigation, it is believed that the three-dimensional gels of the present invention should have all, or substantially all, of the basement membrane proteins in substantially the same ratio as the original tissue basement membrane.

The present invention is described in further detail in connection with the following examples which illustrate or simulate various aspects involved in the practice of the invention. It is to be understood that all changes that come within the spirit of the invention are desired to be protected and thus the invention is not to be construed as limited by these examples.

### EXAMPLES

### Example 1

### Glioblastoma Multiform Xenografts

Human glioblastoma multiform tumor xenografts (U87 cells) were harvested from severe combined immune deficient (SCID) mice. Tumor samples were harvested from both control mice and mice following irradiation to show differences in basement membrane gels following irradiation.

Comparative samples were either suspended in dispase solution, such as from BD Biosciences of San Jose, Calif. USA 95131, at two ml of dispase per gram of tumor, or taken directly to the homogenization step if cellular contamination is not an issue, such as for an autologous use. The dispase treated samples were incubated for 30 minutes at 4° C. in the dispase. It is noted that other enzymatic treatments, chemical treatments, or mechanical removal of the cells might also be used in conjunction with a decellularization step. The dispase treated tissue samples were filtered through a sieve with a 100 μm cell strainer in order to remove contaminating cells. Cell removal was verified under a phase-contrast microscope. Other verification, such as DAPI stain, of the decellularization may be used.

Dispase-treated and non-treated samples were then homogenized in a high salt buffer solution of 0.05M Tris (tris(hydroxymethyl)aminomethane), 3.4M NaCl, 4 μM EDTA (ethylenediaminetetraacetic acid) and 2 μM NEM (N-ethylmaleimide) containing protease inhibitors including 0.001 mg/ml pepstatin, 0.01 mg/ml aprotonin and 0.001 mg/ml leupeptin, each from Sigma-Aldrich Co. of St. Louis, Mo. Other protease inhibitors may include 2 mM sodium orthovanadate (NA₃VO₄) and 1 mM phenylmethylsulfonyl fluoride (PMSF), also available from Sigma-Aldrich Co., or other inhibitors as necessary or desired. The homogenized mixture was washed and centrifuged three times at 7000 g for 15 minutes and the supernatant discarded.

The pellets were then extracted using 2M urea buffer (0.15M NaCl and 0.05 Tris) in a ratio of 1 ml of buffer to each gram of original tissue weight.

The mixture was then stirred continuously for an effective amount of stirring time, e.g., between 6 and 24 hours, and more generally overnight, at 4° C. Stirring might also be done at room temperature, e.g., at about 25° C., although the protein concentration may be reduced. After the effective amount of stirring time the mixture was centrifuged at 14,000 g for 20 minutes, the supernatant was stored, and the pellet was resuspended in 2M urea buffer solution at double the original concentration, i.e., 0.5 ml per gram of original tissue. The pellet was homogenized and centrifuged again at 14,000 g for 20 minutes. The supernatants from both centrifugation steps were collected and maintained at 4° C. and analyzed for protein concentration by performing Bicinchoninic Acid (BCA) protein assays.

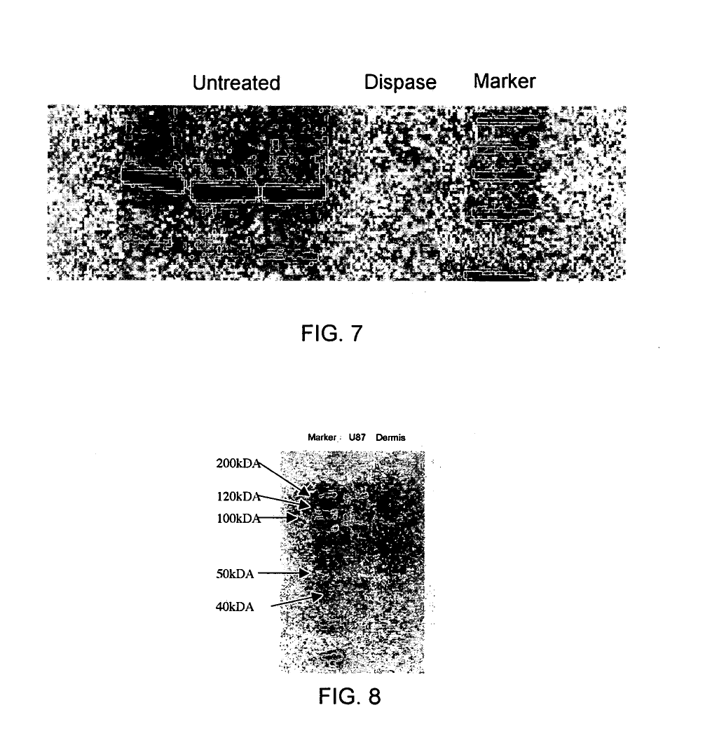

The extraction concentrations ranged between 1.2-2.6 mg/ml and 1.7-1.8 mg/ml for non-irradiated and irradiated U87 tumors, respectively. Referring to FIG. 7, Western blots for the intracellular protein beta actin were performed on dispase treated and untreated extractions to confirm the lack of beta actin in the dispase treated samples. Lane 5 is the molecular weight marker. DAPI nuclear stains were performed on U87 tissues following dispase treatment to verify cell removal. ELISA assays and Western blots were performed in order to detect laminin and collagen components in the glioblastoma tumor basement membrane extract. FIG. 8 shows a collagen type IV Western blot indicating the presence of collagen IV in the irradiated glioblastoma tumor extract and rat dermis basement membrane extractions. The extract comprises the α4 subunit of laminin and collagen type IV. The α4 subunit of laminin has been shown to play an important role in glioblastoma invasion and grade. The α4 subunit of laminin was present at high levels in the U87 and not in a comparative sample of Matrigel. A collagen type IV ELISA assay was performed and showed that there are different relative levels of collagen type IV in extracts from the irradiated and non-irradiated tumors.

Extracted basement membrane proteins in the 2M urea solution were induced to form a stable three dimensional gel by addition of acid to lower the pH from 7.4 to 4. Acetic acid in a 1/150 ratio was used to adjust the pH level, although it is anticipated that other acids may be sufficient to lower the pH and induce gellation. Any method for donating H⁺ ions to the extracted solution is contemplated for use in this invention to induce gellation.

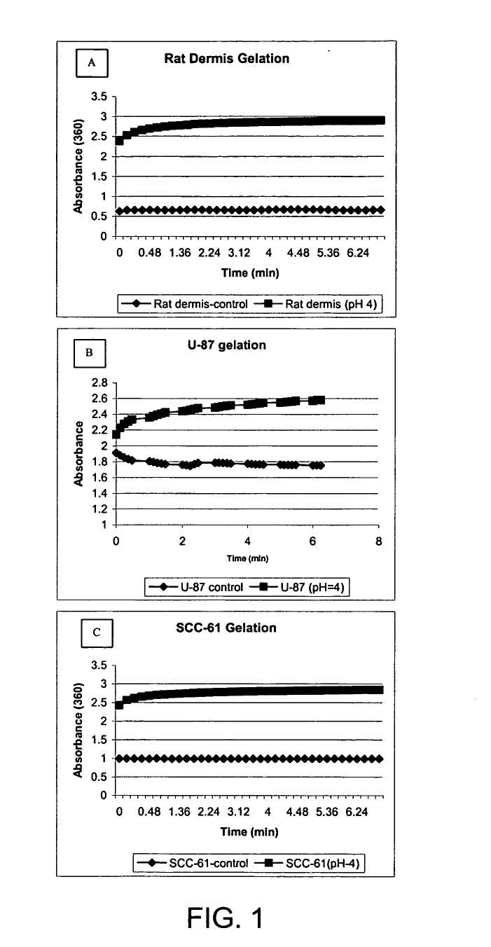

As indicated in FIG. 1, the extracts gelled rapidly following the lowering of the pH and reached the maximum value within about 5 minutes. The gel formation, as a function of light absorbance at 360 nm wavelength, was measured over time using a SPECTRAMAX absorbance microplate reader with SOFTMAX PRO software from Molecular Devices Corporation of Menlo Park, Calif.

If desired, the solution may be sterilized by being dialyzed against 0.25% chloroform in TBS (Tris buffered saline) for a sufficient amount of time, e.g. 10 hours, without affecting the ability to gel. Following dialysis against chloroform, the solution can be dialyzed against multiple changes of TBS to remove chloroform from the solution without affecting the ability to gel. If desired, the 2M urea of the solution may be displaced by being dialyzed against saline without affecting the ability to gel.

The three dimensional gels were washed three times with a buffer solution (pH=7.4) and maintained their 3D structure. Human microvascular endothelial cells (HDMECs) were seeded on the surface of the gels in complete media e.g., Clonetics® brand EGM complete media from Cambrex Bioproducts of East Rutherford, N.J., at a pH of 7.4. The gels were biocompatible and maintained HDMEC growth for greater than 2 weeks.

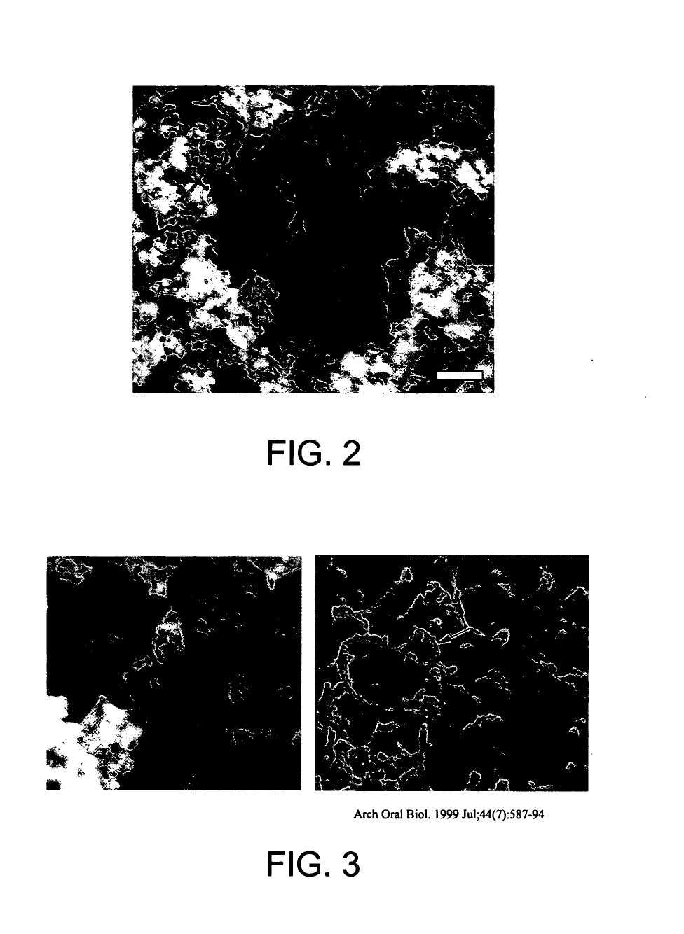

Selected resultant gels were fixed in glutaraldehyde and prepared for scanning and transmission electron microscopy (SEM at 10,000× and TEM at approximately 100,000×, respectively). The gels were observed to have nanostructure similar to what is seen with a basement membrane in vivo as seen in the SEM/TEM photomicrographs of FIGS. 2-4. The SEM images revealed the fiber like structure that holds the gels together. The mean diameters of the fibrils was 83±25 nm which is similar to the 6-100 nm size of basement membrane in vivo, with dependence on the particular tissue or organ location of the basement membrane. Generally, in vivo basement membrane fibrils range in size between 30-150 nm according to E. Adachi, I. Hopkinson and T. Hayashi; Basement-Membrane Stromal Relationships: Interactions Between Collagen Fibrils And The Lamina Densa; International Review of Cytology, 1997; 173:73-156; with a mean fibril diameter of 52+/−28 nm according to G. A. Abrams, C. J. Murphy, Z. Y. Wang, P. F. Nealey, D. E. Bjorling; Ultrastructural Basement Membrane Topography of the Bladder Epithelium, Urological Research, 2003 October; 31(5):341-6. Literature results vary most likely due to actual variation in different organ and tissue locations and alterations due to pathology. Regardless, the gels of this invention generally fall within the range of actual values.

### Example 2

### Squamous Cell Carcinoma (SCC-61) Xenografts

Human squamous cell carcinoma tumor xenografts (SCC-61 cells) were harvested from severe combined immune deficient (SCID) mice and prepared as above in Example 1.

BCA assays were performed and extraction concentrations ranged between 6.7-8.6 mg/ml. ELISA assays and Western blots were performed in order to detect laminin and collagen components in the SCC-61 basement membrane extract. The extract included the β1 and γ1 subunits of laminin. Furthermore, collagen type IV was detected in the extract through an ELISA technique which was used to help identify the protein composition in the SCC-61 extract.

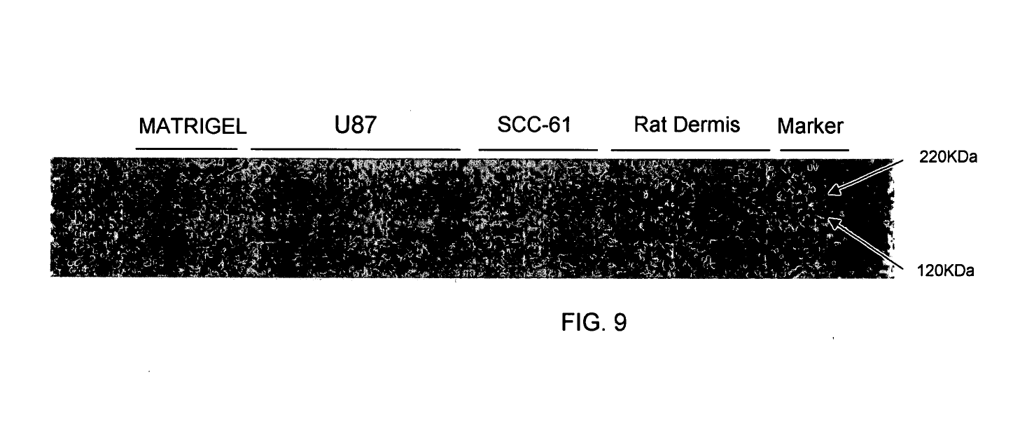

Referring to FIG. 9, a Western blot was performed to further confirm the presence of the γ1 chain of laminin. FIG. 9 shows that the basement membrane extractions from both U87 and SCC-61 xenografts confirming the presence of laminin γ1. MATRIGEL was used as a positive control, indicating the presence of laminin γ1 chain band with a 180 KDa molecular weight, which is the molecular weight at which the γ1 band should be seen.

Extracted basement membrane proteins were induced to form a gel as a result of a change in pH from 7.4 to 4 through the addition of acetic acid as set forth above. The extract began gelling immediately with a change in pH and reached the maximum gellation after approximately 5 minutes.

### Example 3

### Rat Dermis Basement Membrane Extract

Full thickness skin was harvested from rat dorsal skin. Subcutaneous fat was carefully dissected away and the underlying dermis harvested. The basement membrane was extracted as described in Example 1 with all centrifugation steps performed at 4° C. BCA assays were performed and extract concentration was determined to be 10.73 mg/ml. Additional extractions were performed with centrifugation at room temperature (25° C.). These extracts had a concentration of 3.5 mg/ml.

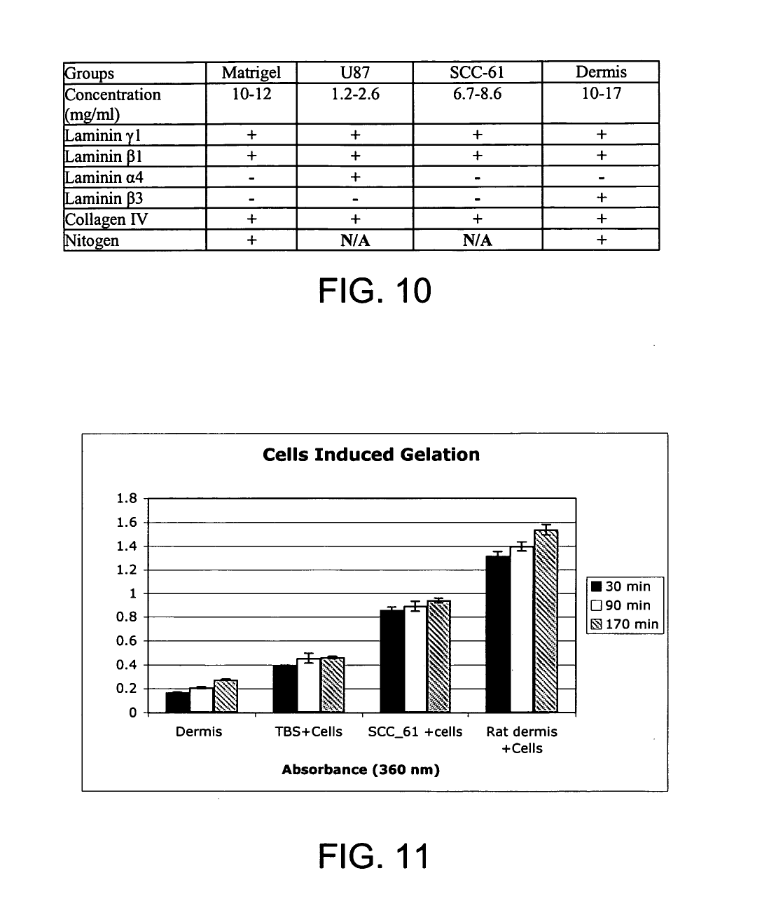

ELISA assays and Western blots were performed in order to detect laminin and collagen components in the rat dermis basement membrane extract. ELISAs indicated that the extract contains the β1 chain of laminin, nidogen, and collagen type IV. Collagen type IV was also detected in the extract through a Western blot. Referring to FIG. 10, ELISAs also indicated the presence of β3, which is not present in any of the other extracts, nor MATRIGEL. Laminin β3 is known to play an important role in physiologic and pathologic phenomenon in the dermis.

Extracted basement membrane proteins (concentration=10.73 mg/ml) gelled when transferred from storage at −20° C. to temperature greater than 5° C. This occurred without altering pH (pH=7.4 throughout the process). The lower concentration extract (3.5 mg/ml) assembled into a three-dimensional gel when pH was decreased from 7.4 to 4 (See FIG. 1), but did not gel when temperature was changed without altering the pH. The gel formation (absorbance at 360 nm wavelength) over time was measured using the absorbance microplate reader (supra). Samples were fixed in glutaraldehyde and imaged by SEM. The temperature gelled samples revealed the fiber structure characteristics of basement membrane in vivo. The mean diameters of the fibrils were 36±12 nm, which is smaller than the acid induced U87 gels, but similar to the 6-100 nm size in basement membrane in vivo.

One hundred and fifty microliters of the low extract solution (concentration=3.5 mg/ml) was also mixed with 350,000 fibroblasts (i.e., cell addition) as counted under a microscope using a hemocytometer. This solution rapidly formed a gel without a change in pH. Absorbance readings of 1.5±0.04 showed that the cell and extract solution formed a gel rapidly. These results, shown in FIG. 11, indicate that extracts can be induced into three dimensional gels by incubation with cells, a process we have termed “cell-induced gellation.” Referring to FIG. 11, the rat dermis basement membrane extracts show increased absorbance upon the addition of cells, and do not form gels otherwise at the temperature, concentration and pH used.

Without wishing to be bound by theory, the cell addition according to this invention causes gelling due to acidic surfaces of the cells added. The basement membrane matrix architecture is generally supported by two major networks: laminin and collagen. Laminin network is self assembled, which means it is comprised primarily of laminin molecules. These polymeric interactions are induced by acidic pH since laminin aggregates in acidic microenvironment created by cellular lipid surfaces. The protein globular conformation correlates with the cellular interfacial acidic pH resulting from the insertion of the protein into a negatively charged lipid vesicles environment. The negatively charged polar heads on the surface of the cells give rise to an electrical surface potential which leads to a decrease in surface pH. The decrease in pH effects laminin globule transition and thus induces its network assembly.

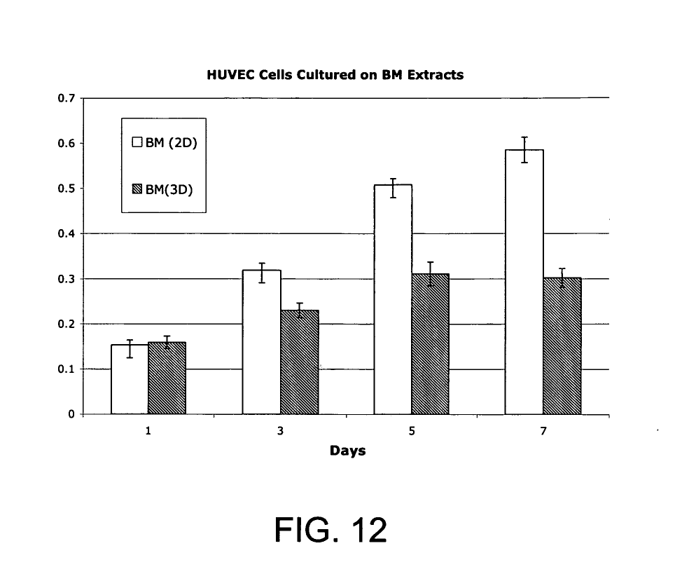

The gels were able to support the growth of both endothelial cells and fibroblasts as either a coating of cell culture dishes or a three-dimensional gel. The two-dimensional culture supported rapid proliferation of both cell types, while lower concentrations supported differentiation (See FIG. 12). This indicates that the substrates are biocompatible and can be used for cell culture studies.

These results indicate that this technique can be used to extract basement membrane proteins from rat dermis. In addition, results show that the concentration of protein extracted decreases when centrifugation steps are performed at 25° C. For highest concentration in the extract all steps should be performed at 4° C. These results also indicate that a threshold concentration exists where, above that concentration, gels will form rapidly when temperature is increased above 4° C. while still at a pH of about 7.4. Below that concentration, pH must be reduced for rapid gel assembly. In addition, these results indicate that basement membrane component gellation can be induced by addition of a sufficient number of cells to the extract solution.

### Example 4

### Bovine Aorta Basement Membrane Extraction

Bovine aortas were received from a local abattoir and stored at minus 80° C. Aortas were thawed and the basement membrane extracted as described in Example 1. Extractions were performed with urea volume to initial tissue weights as described above. BCA assays were performed and the extraction having the 1:11 ratio had a concentration of 7.73 mg/ml while the 0.5:1 ratio concentration was 10.56 mg/ml. The aorta extract rapidly assembled into a 3D gel when pH was changed from 7.4 to 4 by addition of acetic acid.

The basement membrane extraction technique can thus be used to extract basement membrane proteins from bovine aorta. The extracts rapidly assemble into 3D gels following a reduction in pH. Concentration of the membrane extract solution can be increased by reducing the ratio of urea volume to original tissue weight.

Thus, the results of the above examples indicate that: the present invention can be used to extract basement membrane proteins from a variety of tissues; the protein extract can be induced to form a gel by changes in pH, temperature, concentration, or by an addition of cells; a brief treatment with dispase can be used to remove cellular contaminants from the tissue and the resultant extract is substantially free of cellular contamination; the components of the gels are unique and are distinct from currently available products; the gels obtained through pH lowering maintain their structure when the pH is raised again to 7.4; the gels are biologically active and biocompatible, supporting growth of endothelial cells and fibroblasts; and that gels can also be induced to form by incubation with cells in the absence of other environmental changes.

Initiation of gellation of the supernatant solution depends on its protein concentration. Gellation can be controlled by determining the protein concentration, such as by BCA assays, and if necessary, controlling the concentration of the original tissue weight to the volume of solvent or diluent, e.g., 2M urea, and either lowering the pH if the protein concentration is below a critical level, or raising the temperature if the protein concentration is above the critical level. The critical concentration level is currently assumed to be about 5 mg/ml although exact figures may be later established by further experimentation. The threshold pH is currently assumed to be about 4.0 although exact figures may be later established by further experimentation.

The supernatant basement membrane extracts will gel when the pH is reduced from around 7.0 to around 4.0 when below the critical concentration of about 5 mg/ml. Above the critical concentration, the supernatant basement membrane extracts will assemble into a three-dimensional gel at a pH of about 7.0 when brought to room temperature, i.e., about 25° C. Above the critical concentration, gellation can still be induced by decreasing the pH, such as to about 4.

As seen in FIG. 1, the graphs of gellation, as measured by the microplate reader (supra.) for absorbance at 360 nm, versus time starting at the change from pH 7.4 to pH 4.0, show that gellation begins upon a change in pH of the supernatant in less than 5 minutes.

As seen in FIG. 2, upon gellation, the extracted basement membrane components from a U87 glioblastoma xenograft develop into three dimensional gels with a nanostructure similar to the basement membrane in vivo. The bar in the photomicrograph is 1 micron. Transmission and scanning electron microscopy (TEM and SEM, respectively) were performed to characterize the structure of the gels of the present invention and compare them to the known structure of basement membrane found in vivo. The SEM images at 10,000× magnification reveal the fiber-like structure that holds the gels together. The mean diameter of the fibrils was 83±25 nm which is similar to their size in basement membrane in vivo. Referring to FIG. 2 and FIG. 3A, SEM images (also U87) reveal a meshwork structure of fine fibers within the gel. The diameter of the fibrils is on a similar scale (about 6-100 nm) to that found in published reports for basement membrane in vivo as shown in FIG. 3B.

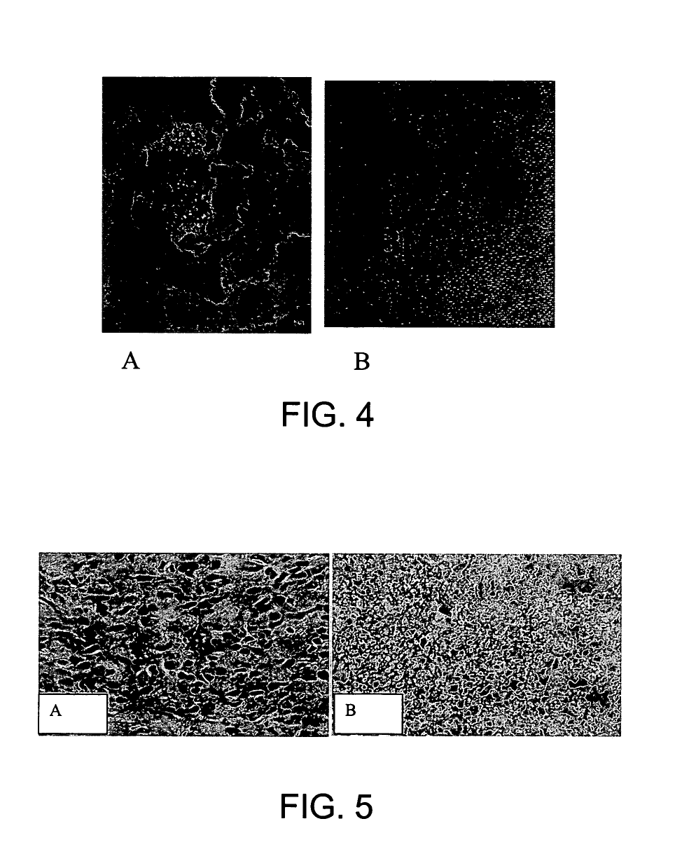

Referring to FIG. 4A, TEM images of the extracted gels (also U87) show a diffusively flocculent material. This structure is similar to what is seen in TEMs of basement membrane underlying cells in tissue samples as seen by TEM. The structure of the gels is distinctly characteristic of basement membrane in vivo as shown in FIG. 4B.

Referring to FIG. 5, Periodic Acid Schiff (PAS) staining was performed on original tissue samples (also U87) and extracted gels to highlight matrix architecture. The gel of FIG. 5B had a similar staining pattern to the matrix (non-cellular) component of the original tissue as seen in FIG. 5A. These stains combined with the electron microscopy show that this procedure allows isolation of membrane gels with similar structure to basement membranes in vivo.

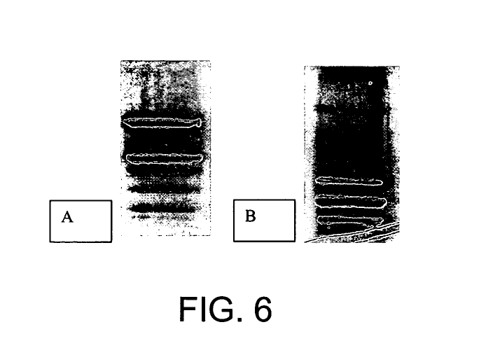

As seen in FIG. 6, sodium dodecyl sulphate polyacrylamide gel electrophoresis (SDS-PAGE) analysis shows the high molecular weight components (also U87) present in the isolation procedure. The basement membrane components in the extracted gels illustrated in FIG. 6A are distinct from the proteins present in a commercial MATRIGEL preparation as illustrated in FIG. 6B.

Western blot analysis has verified the presence of the gamma 1 chain of laminin in extracts of the present invention. Laminin is an important basement membrane protein and the isoforms present vary with tissue type. As indicated by the dermis and U87 results, laminin isoforms are present in the extracts of this invention that are not typically present in commercially available basement membrane complexes. Thus, it is especially desirable to utilize selectable tissue as the precursor to the three dimensional gels according to the present invention.

By attaining the replicated or duplicated basement membrane gels from unique selectable tissue sources, several advantages occur. Gels isolated with this procedure can be used as basic research tools for studying structure-function interactions in both pathologic and normal tissues. These studies may assist in identifying cues present in the basement membrane that influence normal and abnormal tissue physiology. Gels of the present invention can be relevant to particular tissues of interest, and used for studies of tumor invasion, angiogenesis, cell adhesion, pro- and anti-therapeutics, and response to therapeutics.

Because the gels are derived from tissues specifically selected, several potential therapeutic applications for regenerative medicine applications result. The angiogenic, chemotactic, and tissue specific differentiation properties of gels extracted using this procedure make them ideal candidates for use in regenerative medicine. Gels according to the present invention could be used as a tissue engineering scaffold, to promote wound healing or tissue regeneration, or as a bulking agent. Because the gels of the present invention can be decellularized, i.e., made to lack cellular contaminants, they can be used without a high incidence of rejection reactions. If used autologously, as discussed above, the original tissue need not necessarily be decellularized. The decellularized gels could be prepared from cadavers, human donors, or animal tissues. Use of the patient's own tissue, or decellularized gels in general, will greatly reduce the risk of rejection. Tests have shown that sterilization by chloroform dialysis does not affect the gellation of the present invention.

Extracellular and basement membrane materials may also be used as drug delivery systems. For example, DNA, small molecules, or proteins encapsulated in the gels can be injected into a tissue location for localized and sustained delivery. Gels developed with this method might also be used in studies of stem cell differentiation and in feeder free stem cell culture systems where use of animal serums should be avoided.

Also, basement membrane fragments have revealed important peptide sequences that promote cellular adhesion. Newly selected gels could lead to the identification of new and important cell adhesion sequences. Further, proteolytic fragments from basement membrane and extracellular matrix are important cancer therapeutics. Studies with these gels could identify new anti-angiogenic and anti-tumor therapeutics.

Due to the ability of cells to initiate protein gellation, these gels could be used as vehicles for cell delivery. These cells could be autologous, xenogenic, or allogenic and could be used as cellular therapies or could be used in animals models to study basic and pathologic biologic phenomena. Injectable approaches could be used where cells are suspended within the extracts and injected into a patient where the cells then induce formation of gels in situ.

Thus, a gel according to the present invention may have many therapeutic or research uses including but not limited to: a coating for cell culture substrates; as a substrate for stem cell culture; as a model for studying cell migration, invasion, and angiogenesis in vitro; in the treatment of neurodegenerative diseases; to study differences in tumor biology between normal and genetically modified tissue stroma; to promote endothelialization of vascular grafts, blood vessels, or tissue engineered vascular grafts; to improve healing of diabetic ulcers; as a treatment for wrinkles, hair loss; to stimulate wound healing; to stimulate angiogenesis in ischemic tissues; as a scaffold for tissue regeneration; and for in vitro organogenesis.

Along with the other benefits of the present invention, it is believed that the cost of production of gels developed according to the present invention is low. Also, sterilization does not alter the ability of the method to assemble a three dimensional gel. Further, local decreases in pH occurring with some currently used biomaterials may also be avoided by equilibrating the gels initially in physiologic pH, e.g., the gels of the present invention can be maintained at pH=7.4, or utilizing other methods for initializing gellation such as set forth herein.

While certain exemplary embodiments have been put forth to illustrate the present invention, these embodiments are not to be taken as limiting to the spirit or scope of the present invention which is defined by the appended claims.

We claim:

1. A method of obtaining a gel structure including a protein extract from a tissue, the method comprising:
selecting a tissue for extraction of basement-membrane proteins;
obtaining a solution of the basement membrane components from the tissue;
gelling the extracted basement membrane by at least one of lowering the pH of the solution of the extracted basement membrane components or adding cells to the solution of the extracted basement membrane.

2. The method of claim 1, further comprising lowering the pH to below about 5.

3. The method of claim 1, further comprising lowering the pH to about 4.0.

4. The method of claim 1, wherein the gel has basement membrane components in a same ratio as a basement membrane of the selected tissue.

5. The method according to claim 1, wherein the gel forms within about 5 minutes upon lowering the pH.

6. The method of claim 1, further comprising decellularizing the tissue to achieve a decellularized basement membrane that is at least substantially free of cellular contaminants.

7. The method of claim 6, further comprising grinding the tissue or treating the tissue with an enzyme or a chemical.

8. The method of claim 7, wherein the enzyme comprises dispase.

9. The method of claim 1, wherein obtaining a solution of the basement membrane components comprises grinding the tissue in a salt solution in the presence of a protease inhibitor.

10. The method of claim 1, wherein obtaining a solution of the basement membrane components comprises extracting basement membrane components in a urea solution.

11. The method of claim 10, wherein the solution comprises a concentration greater than about 0.1 mg/ml.

12. The method of claim 10, further comprising stirring the tissue sample in a urea solution for an effective time at between about 0° to about 25° C.

13. The method of claim 1, further comprising performing increasing the pH of the gel to about 7.0 to about 7.4.

14. The method of claim 1, wherein the tissue is selected from human glioblastoma multiform tumor xenografts, human squamous cell carcinoma tumor xenografts, rat dermis, and bovine aorta.

15. A gel made according to the method of claim 1.

16. A method of obtaining a gel structure including a protein extract from a tissue, the method comprising:
selecting a tissue for extraction of basement-membrane proteins;
removing cells of the selected tissue to achieve a decellularized basement membrane free of cellular contaminants;
extracting basement membrane components from the decellularized basement membrane; and
gelling the extracted basement membrane.

17. The method of claim 16, wherein gelling the extracted basement membrane comprises lowering a solution pH to below about 5.

18. The method of claim 16, wherein gelling the extracted basement membrane comprises increasing a solution temperature above about 30° C.

19. The method of claim 18, wherein a solution of the basement membrane components comprises a concentration greater than about 5 mg/ml.

20. The method of claim 16, wherein gelling the extracted basement membrane comprises adding cells to a solution of the basement membrane components.

21. The method of claim 16, wherein removing cells of the selected tissue comprises grinding the tissue or treating the tissue with an enzyme or a chemical.

22. The method of claim 16, wherein extracting basement membrane components from the tissue comprises grinding the tissue in a salt solution in the presence of a protease inhibitor.

23. The method of claim 16, wherein the extracted basement membrane components are placed in a urea solution.

24. A method of obtaining a gel structure including a protein extract from a tissue, the method comprising:
selecting a tissue for extraction of its basement-membrane proteins;
homogenizing the basement membrane in a salt solution containing a protease inhibitor;
extracting basement membrane components of the homogenized basement membrane in a high molar urea solution;
gelling the extracted basement membrane components in the urea to attain a gel structure by lowering the pH of the solution or adding cells to the solution.
